(19) United States
(12) Patent Application Publication
(10) Pub. No.: US 2020/0015752 A1
(43) Pub. Date: Jan. 16, 2020

(54) TEXTILE UTILIZING CARBON NANOTUBES

(71) Applicant: John R Baxter, Greenville, SC (US)

(72) Inventor: John R Baxter, Greenville, SC (US)

(21) Appl. No.: 16/035,492

(22) Filed: Jul. 13, 2018

(51) Int. Cl. A61B 5/00; A61B 5/05; D04H 13/00; G01R 19/00

(52) U.S. Cl. CPC A61B 5/6892; A61B 5/05; D10B 2509/00; G01R 19/0092; D10B 2101/122; D04H 13/00

(57) ABSTRACT

A mattress utilizing carbon nanotube alone or in combination with fibrous material in the form of a textile. The textile having semi conductive properties to allow for grounded ion exchange to improve electromagnetic hygiene, detect biological processes, and/or evaluate a health condition.

### FIELD OF THE INVENTION

The present invention generally relates to carbon nanotube layers, and more particularly to carbon nanotube layers configured for contacting a biological body.

### BACKGROUND

Since the Industrial Age, protecting electronics from Electro Static Discharge (ESD), Electro Magnetic Frequency (EMF) and aberrant fluctuations in charge is considered a best practice, but until recently, little thought has been given to biological effects EMF and aberrant fluctuations in charge existing primarily as Subliminal or “below threshold” stimuli have on the human organism. Recent studies continue to emerge illustrating that in addition to protection from ESD, placing a human in conductive contact with the electrically negative potential of the Earth has measurable physiological impact and consequence to health.

Discovery of new and novel ways to “cleanse” ourselves of the injurious and deleterious stimuli of EMF and aberrant electrical charge has become the basis of a new field of study, “electromagnetic hygiene.” Materials may be deployed in such a manner as to harness the Earth's electrically negative potential to cancel and/or reduce electric fields around the human body, as well as attenuate oxidative stress and reduce damage to the body from positively charged reactive oxygen species (free radicals).

Efforts continue, therefore, to develop materials which facilitate electromagnetic hygiene with greater resilience, durability, and utility.

### SUMMARY

To overcome limitations in the prior art, and to overcome other limitations that will become apparent upon reading and understanding the present specification, various embodiments of the present invention disclose a method and apparatus for securing a bezel to a rail nut spacer contained in a slot extending across a housing.

In accordance with one embodiment of the invention a sleep system includes a first textile including carbon nanotube material, wherein the textile is configured to exchange free-moving electrons with an organic body.

In another embodiment of the invention, a method includes placing an organic body on a first textile, the first textile including carbon nanotube material, and exchanging free-moving electrons between the organic body and the first textile.

In another embodiment of the invention, a method includes placing an organic body on a first textile, the first textile including carbon nanotube material; and detecting the exchange of free-moving electrons between the organic body and the first textile.

### BRIEF DESCRIPTION OF THE DRAWINGS

Various aspects and advantages of the invention will become apparent upon review of the following detailed description and upon reference to the drawings in which:

FIG. 1 illustrates a carbon hexagonal lattice;

FIG. 2 illustrates an armchair carbon hexagonal lattice;

FIG. 3 illustrates a 3-layer armchair carbon hexagonal lattice;

FIG. 4 illustrates a cross-sectional view of a carbon nanotube and/or carbon nanotube composite textile utilized in a sleep system;

FIG. 5 illustrates a cross-sectional view of a carbon nanotube and/or carbon nanotube composite textile utilized in a sleep system;

FIG. 6 illustrates a sleep system connected to a relatively greater negative electrical potential;

FIG. 7 illustrates a sleep system connected to a medical device capable of detecting biological processes;

FIG. 8 illustrates a sleep system connected to a system capable of evaluating a health condition of a body;

FIG. 9 illustrates a carbon nanotube fiber formed by a plurality of carbon nanotubes;

FIG. 10 illustrates first and second carbon nanotube fibers forming a carbon nanotube strand;

FIG. 11 illustrates a plurality of carbon nanotube fibers forming a carbon nanotube strand;

FIG. 12 illustrates a plurality of carbon nanotube fibers forming a carbon nanotube strand;

FIG. 13 illustrates a carbon nanotube textile formed by a plurality of carbon nanotube fibers and/or strands;

FIG. 14 illustrates a carbon nanotube textile formed by a plurality of carbon nanotube fibers and/or strands;

FIG. 15 illustrates a textile formed of a fibrous material; and

FIG. 16 illustrates a textile formed of a fibrous material.

### DETAILED DESCRIPTION

Generally, the various embodiments of the present invention are applied to a carbon nanotube apparatus for facilitating electromagnetic hygiene, including, but not limited to, one or more of controlling aberrant fluctuations in charge of the body, causing static electric and/or electro-static discharge of the body, reducing electric fields around the body, cancelling electric fields around the body, placing the body in conductive contact with the Earth, attenuating oxidative stress in the body, allow a flow on negatively charged ions in the body, the exchanging of ions by removing positive electrons and introducing negative electrons, neutralizing and/or reducing free radicals in the body, and/or electro-magnetic field (“EMF”) protection. The body may refer to the human body, or any other species or organic body which may derive a benefit from electromagnetic hygiene (e.g., canines).

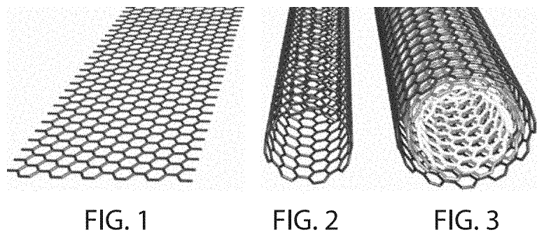

Carbon may be capable of being formed in many allotropes due to its valency, including graphite, diamond, charcoal, carbon nanotubes, nanobuds, nanoribbons, and fullerenes. Generally, each carbon allotrope may be formed by a repeating pattern of six (6) carbon atoms (a carbon molecule), which, when repeated may have a honeycomb, or hexagonal lattice appearance, as exemplified in FIG. 1. Adjacent carbon molecules generally may share at least two carbon atoms in the hexagonal lattice. The hexagonal lattice of FIG. 1 is illustrated with a number of interconnected hexagonal shapes, where each line represents a bond or link between adjacent carbon atoms and each carbon atom is positioned at the intersection of three (3) lines.

While graphite is formed of a plurality of layers of carbon hexagonal lattice, whether aligned in parallel or otherwise, a single layer of carbon hexagonal lattice may generally referred to as graphene. Graphene may be rolled at specific and discrete angles, or chiral, to form nanotubes, which rolling angle and radius may determine the properties of the nanotube. For example, as exemplified in FIG. 2, a carbon hexagonal lattice may be rolled about an axis perpendicular to at least one of the carbon atom bonds to form an armchair carbon nanotube as exemplified in FIG. 2. While the nanotube of FIG. 2 is exemplified with approximately 14 rows of carbon molecules, a person of ordinary skill in the art will appreciated that fewer or greater numbers of rows of carbon molecules may be possible (e.g., 2 or more rows of carbon molecules).

In another example, a carbon hexagonal lattice may be rolled about an axis parallel to at least one of the carbon atom bonds to form a zigzag carbon nanotube. In another example, a plurality of hexagonal lattice may be rolled to form a multilayered armchair carbon nanotube, as exemplified in FIG. 3. In another example, a plurality of hexagonal lattice may be rolled to form a multilayered zigzag carbon nanotube. A person of ordinary skill in the art will appreciated that fewer or greater numbers of rows of carbon molecules may be possible in each of these examples.

The carbon nanotube apparatus may be formed purely of carbon nanotubes (e.g., graphene carbon nanotube material), or may be formed of carbon nanotubes in combination with any fibrous material, collectively referred to as a carbon nanotube composite. For example, a carbon nanotube composite may be formed of seventy percent (70%) carbon nanotube and thirty percent (30%) of one or more other materials (e.g., polyester). In another example, a carbon nanotube composite may be formed of thirty percent (30%) carbon nanotube and seventy percent (70%) of one or more other materials (e.g., cotton). In another example, a carbon nanotube composite may be formed of fifty percent (50%) carbon nanotube and fifty percent (50%) of one or more other materials (e.g., 25% cotton and 25% polyester). A person of ordinary skill in the art will appreciate that various other combinations may be possible utilizing known fibrous materials.

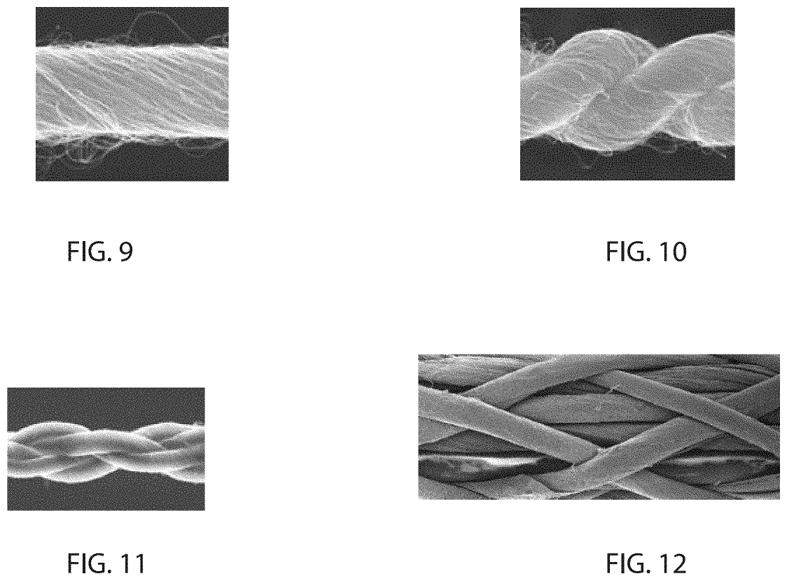

Carbon nanotubes and/or carbon nanotube composite may be formed into a fiber, such that a plurality of carbon nanotube and/or carbon nanotube composite fibers may be weaved, entwined, or otherwise fabricated to form a textile (e.g., as exemplified in FIGS. 9-12). The textile may be used in a manner so that the textile may come into contact with the body in order to facilitate electromagnetic hygiene as herein described. For example, the carbon nanotube and/or carbon nanotube composite textile may take the form of bedding material (e.g., a textile configured for placement over the body), a mattress cover (e.g., a textile configured for placement under the body), and/or a mattress quilting (e.g., a textile configured to enclose a top portion of a mattress). In another example, the carbon nanotube and/or carbon nanotube composite textile may be configured in one or more of a hat, a jacket, a shirt, trousers, a sock, or any other article of clothing or article meant to be worn on the body. In another example, the carbon nanotube and/or carbon nanotube composite textile may be configured in one or more of a rug, carpet, or other floor covering which contacts the body. In another example, the carbon nanotube and/or carbon nanotube composite textile may be configured in one or more of a blanket, a seat covering, or other article of furniture which contacts the body when in use thereby. A person of ordinary skill in the art will appreciate that various other applications are possible wherein utilizing carbon nanotube and/or carbon nanotube composite textile may facilitate greater electromagnetic hygiene.

Carbon nanotubes and/or carbon nanotube composites may enjoy a number of advantages over other materials and/or composites previously known for use to facilitate electromagnetic hygiene. For example, the carbon nanotube structure may be highly stable due to its tightly packed, periodic array of carbon atoms and sp² orbital hybridization. In another example, the carbon nanotube may be resistant to corrosion when put into contact with the body (e.g., due to pH of the body) and/or contaminants on the surface of the body (e.g., lotions) and/or contaminants excreted by the body (e.g., sweat). In another example, the carbon nanotube may be resistant to corrosion when washed (e.g., during a washing cycle of a washing machine), and further may be resistant to chemicals used for this or any other purpose. In another example, carbon nanotubes may have increased durability and longevity (e.g., greater than 6, 9, 12, or 18 months) over other materials (e.g., silver) because of the resistance to corrosion.

In another example, the sp² orbital hybridization of the carbon nanotube structure may be key to the half-filled band that permits free-moving electrons for facilitating conductivity (e.g., 200,000 cm-2v-1s-1, greater than copper or any other material), and may allow one or more of electromagnetic hygiene, detection of a biological process (e.g., vital signs) in the body, and/or evaluating a health condition of the body. In another example, a carbon nanotube may be highly durable because it can self-repair holes in its hexagonal lattice when exposed to molecules containing carbon (e.g., hydrocarbons). In another example, the carbon nanotubes structure may be weak in compression and torsion, which may allow it to be highly flexible during use with the body.

In another example, the carbon nanotube structure may be capable of conducting heat and may be used for heating and/or cooling of the body (e.g., during sleep). In another example, the carbon nanotube and/or carbon nanotube composite textile may be breathable, in that there may be air pockets disposed intermittently between fibers and/or strands of the carbon nanotube, fibrous material, and/or composite material. The breathable composition herein described may allow cooling of the body and/or dissipation of heat.

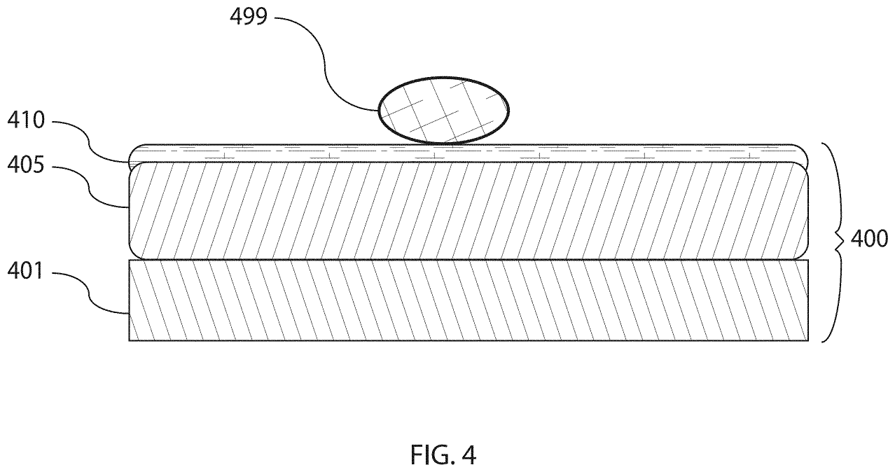

FIG. 4 illustrates a cross-sectional view of a carbon nanotube and/or carbon nanotube composite textile 410 utilized in a sleep system 400. Sleep system 400 may be configured with one or more of a foundation (e.g., box spring 401), a mattress (e.g., formed of a base element 405), and a mattress covering (e.g., carbon nanotube and/or carbon nanotube composite textile 410).

Base element 405 may be configured on box spring 401. For example, the overall length and width dimensions of base element 405 and box spring 401 may be substantially similar. In another example, base element 405 may be configured to interconnect with box spring 401 (e.g., via zip, clip, buckle, hook and loop, seam or other known fastening means). Textile 410 may be configured on base element 405. For example, the overall length and width dimensions of textile 410 and base element 405 may be substantially similar. In another example, textile 410 may be configured to interconnect with base element 405 (e.g., via zip, clip, buckle, hook and loop, seam or other known fastening means).

A body 499 may lay on top of sleep system 400 (e.g., on top of textile 410). For example, body 499 may contact textile 410 at at least one position. In another example, body 499 may come into field contact with textile 410, such that an electric and/or magnetic field of body 499 may be close enough to textile 410 to interact therewith and/or enable the transfer of electrons therebetween.

Nevertheless, a person of ordinary skill in the art will appreciate that body 499 may be likely to contact textile 410 at many positions along the body. While it is not shown in FIG. 4, it may be generally understood that the weight of body 499 will cause compression of textile 410 and/or base element 405, such that body 499, to some extent, will sink into textile 410 and/or base element 405 increasing the surface area of contact between body 499 and textile 410. Thus body 499, upon laying on top of sleep system 400 may come into substantial contact with textile 410. The substantial contact between body 499 and textile 410 may enable body 499 to experience improved electromagnetic hygiene. In another example, textile 410 may be configured of any one or more of the fibers, strands, textile and/or composite fiber, strand, or textile as exemplified in FIGS. 9-16. In another example, textile 410 may be configured of any fiber, strand, and/or textile known to a person of ordinary skill in the art.

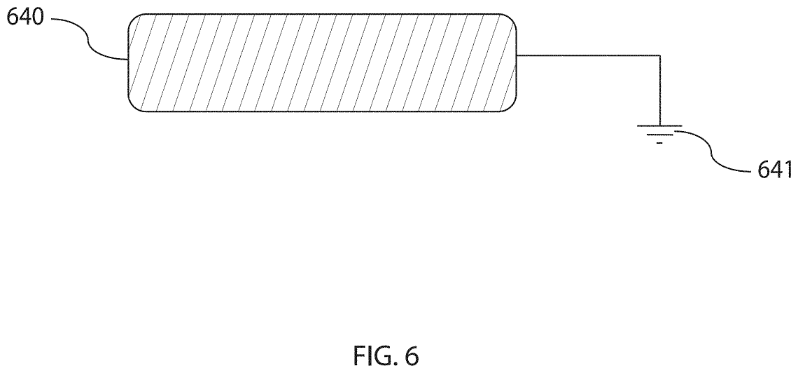

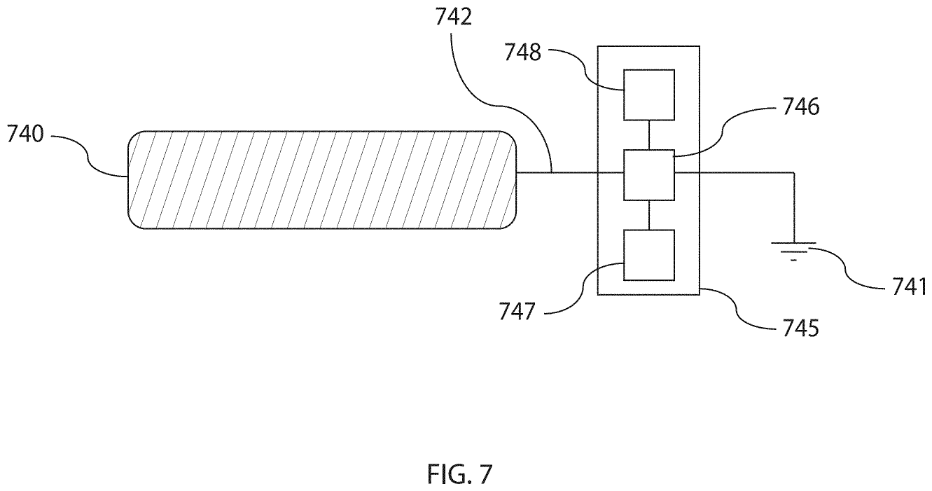

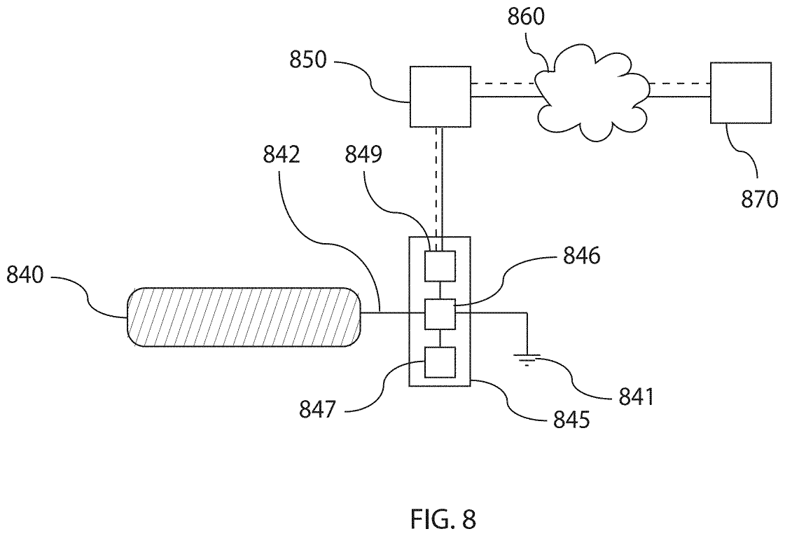

Furthermore, textile 410 may be mechanically and conductively connected to other components. For example, textile 410 may be connected to a relatively greater negative electrical potential, such as electrical ground (e.g., as exemplified in FIG. 6) to further improve electromagnetic hygiene. For example, connection to a relatively greater negative electrical potential may enable EMF protection, exchange of electrons, and/or any of the benefits of electromagnetic hygiene as herein described. In another example, textile 410 may be connected to a medical device capable of detecting biological processes (e.g., as exemplified in FIG. 7). In another example, textile 410 may be connected to a system capable of evaluating a health condition of the body (e.g., as exemplified in FIG. 8).

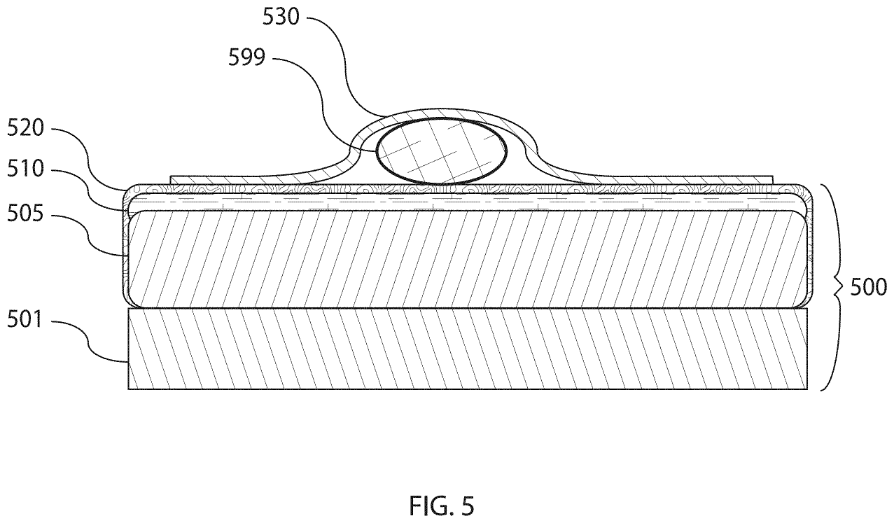

FIG. 5 illustrates a cross-sectional view of a carbon nanotube and/or carbon nanotube composite textile utilized in a sleep system 500. Sleep system 500 may be configured with one or more of a foundation (e.g., box spring 501), a mattress (e.g., formed of a base element 505 and a top element 510), a mattress covering (e.g., mattress cover 520), and/or a bedding material (e.g., sheet 530).

Mattress cover 520 may be configured to extend around and/or protect the mattress. For example, mattress cover 520 may extend over at least one surface of the mattress. In another example, mattress cover 520 may extend over substantially all surfaces of the mattress except for a surface which contacts the box spring 501. In another example, mattress cover 520 may be configured to interconnect with the mattress (e.g., via zip, clip, buckle, hook and loop, seam or other known fastening means). In another example, mattress cover 520 may be configured to snugly fit over the mattress by means of in-seam elastic around the perimeter edge. In another example, mattress cover 520 may be placed over the mattress with no securement. In another example, mattress cover 520 may be configured of any one or more of the fibers, strands, textile and/or composite fiber, strand, or textile as exemplified in FIGS. 9-16. In another example, mattress cover 520 may be configured of any fiber, strand, and/or textile known to a person of ordinary skill in the art.

A body 599 may lay on top of sleep system 500 (e.g., on top of mattress cover 520), and a sheet 530 may be placed over body 599 and/or mattress cover 520. For example, body 599 may contact mattress cover 520 at at least one position. In another example, body 599, upon laying on top of sleep system 500 may come into substantial contact with mattress cover 520. In another example, body 599, upon use of sheet 530, may contact sheet 530 at at least one position. In another example, body 599, may come into substantial contact with sheet 530. In another example, mattress cover 520 and/or sheet 530 may substantially conform to the shape of body 599. In another example, body 599 may be substantially enclosed by mattress cover 520 and sheet 530.

In the instant embodiment, mattress cover 520 may be composed of one or more of carbon nanotubes and/or fibrous material. Thus, direct and/or field contact between body 599 and mattress cover 520 may enable body 599 to experience improved electromagnetic hygiene. In an alternative embodiment, sheet 530 may be composed of one or more of carbon nanotubes and/or fibrous material. Thus, direct and/or field contact between body 599 and sheet 530 may enable body 599 to experience improved electromagnetic hygiene. In an alternative embodiment, mattress cover 520 and sheet 530 may be composed of one or more similar or different amounts of carbon nanotubes and/or fibrous material. Thus, direct and/or field contact between either body 599 and mattress cover 520 or body 599 and sheet 530 may enable body 599 to experience similar or different amounts of electromagnetic hygiene.

Furthermore, mattress cover 520 and/or sheet 530 may be mechanically and conductively connected to other components. For example, mattress cover 520 and/or sheet 530 may be connected to a relatively greater negative electrical potential, such as electrical ground (e.g., as exemplified in FIG. 6) to further improve electromagnetic hygiene. In another example, mattress cover 520 and/or sheet 530 may be connected to a medical device capable of detecting biological processes (e.g., as exemplified in FIG. 7). In another example, mattress cover 520 and/or sheet 530 may be connected to a system capable of evaluating a health condition of the body (e.g., as exemplified in FIG. 8).

FIG. 6 illustrates a component 640 of a sleep system (e.g., sleep system 500 of FIG. 5) mechanically and conductively connected to a relatively greater negative electrical potential (e.g., ground 641). Component 640 may represent any carbon nanotube and/or carbon nanotube composite textile utilized in the present invention. For example, component 640 may represent textile 410 of FIG. 4. In another example, component 640 may represent mattress cover 520 of FIG. 5. In another example, component 640 may represent sheet 530 of FIG. 5. In another example, component 640 may represent both mattress cover 520 and sheet 530 (insofar as they are both mechanically and conductively connected to ground 641).

FIG. 7 illustrates a component 740 of a sleep system (e.g., sleep system 400 of FIG. 4) mechanically and conductively connected to a medical device 745 capable of detecting biological processes. Component 740 may represent any carbon nanotube and/or carbon nanotube composite textile utilized in the present invention. For example, component 740 may represent textile 410 of FIG. 4. In another example, component 740 may represent mattress cover 520 of FIG. 5. In another example, component 740 may represent sheet 530 of FIG. 5. In another example, component 740 may represent both mattress cover 520 and sheet 530 (insofar as they are both mechanically and conductively connected to the medical device 745).

Medical device 745 may include a processor 746 for sending and/or receiving control signals. For example, the presence of a body (e.g., body 499 of FIG. 4) on component 740 may cause the exchange of electrons from the body to component 740, from component 740 to wire 742, and/or from wire 742 to processor 746. Thus, processor 746 may be configured to detect the transfer of free-moving electrons from the body. In another example, processor 746 may collect and store signals from the body in a memory 747. In another example, processor 746 may use signals from the body to calculate other useful indicia (e.g., heart rate), which may be stored in memory 747.

In another example, processor 746 may receive control signals from a user interface 748 (e.g., such as a keypad, a touch screen, or other interface means). The user interface 748 may enable a user to access data and/or signals stored in memory 747. In another example, user interface 748 may enable a user to program a heat level of component 740 (e.g., with additional power, if needed, being provided from a power source, such as a traditional wall outlet). In another example, medical device 745 may be mechanically and conductively connected to a relatively greater negative electrical potential (e.g., ground 741).

FIG. 8 illustrates a component 840 of a sleep system mechanically and conductively connected to a system capable of evaluating a health condition of a body. Component 840 may represent any carbon nanotube and/or carbon nanotube composite textile utilized in the present invention. For example, component 840 may represent textile 410 of FIG. 4. In another example, component 840 may represent mattress cover 520 of FIG. 5. In another example, component 840 may represent sheet 530 of FIG. 5. In another example, component 840 may represent both mattress cover 520 and sheet 530 (insofar as they are both mechanically and conductively connected to the system capable of evaluating a health condition of a body).

The system capable of evaluating a health condition of a body may include a local device 845 with a processor 846 for sending and/or receiving control signals. For example, the presence of a body (e.g., body 499 of FIG. 4) on component 840 may cause the exchange of electrons from the body to component 840, from component 840 to wire 842, and/or from wire 842 to processor 846. Thus, processor 846 may be configured to detect the transfer of free-moving electrons from the body. In another example, processor 846 may collect and store signals from the body in a memory 847. In another example, processor 846 may use signals from the body to calculate other useful indicia (e.g., heart rate), which may be stored in memory 847.

In another example, processor 846 may send to and receive signals (e.g., transmittal of data corresponding to the exchange of free-moving electrons) from a communication module 849. The communication module 849 may communicate (i.e., send and receive data signals) with a local area network provided by a modem or similar device 850 (e.g., via wired connection, Bluetooth, wifi, or other equivalent means). The modem or similar device 850 may be configured to communicate (e.g., wirelessly or by wired connection) with a remote electronic device 870 (e.g., via the internet 860). Thus, for example, remote electronic device 870 may be capable of receiving signals from the body. In another example, remote electronic device 870 may be capable of accessing data stored in memory 847. In another example, remote electronic device 870 may be capable of sending signals to processor 846. In another example, local device 845 may be mechanically and conductively connected to a relatively greater negative electrical potential (e.g., ground 741).

Remote electronic device 870 may be accessible to a medical professional, licensed contractor, or other party with permission to access data signals detected by processor 846 and/or data signals stored in memory 847. Thus, for example, data signals may be used by said professional to evaluate a health condition of the body. In another example, component 840 and remote electronic device 870 may be configured in the same building (e.g., a hospital). In another example, component 840 and remote electronic device 870 may be configured remotely (e.g., component 840 may be in a private residence and remote electronic device may be in a doctor's office).

FIG. 9 illustrates a carbon nanotube fiber formed by a plurality of carbon nanotubes. The nanotubes may be tightly packed and/or spun together into a helical arrangement to increase strength, conductivity, and other properties of the carbon nanotube fiber. FIG. 10 illustrates first and second carbon nanotube fibers, each formed by a plurality of carbon nanotubes. Each fiber may be formed of tightly packed and/or spun nanotubes. Further, the first and second fibers may be tightly packed and/or spun together into a helical strand arrangement to increase strength, conductivity, and other properties of the carbon nanotube strand. FIGS. 11 and 12 illustrate increasingly complex carbon nanotube strands, having greater numbers of carbon nanotube fibers. A person of ordinary skill in the art will appreciate that various other fiber and strand configurations may be possible to achieve desired characteristics.

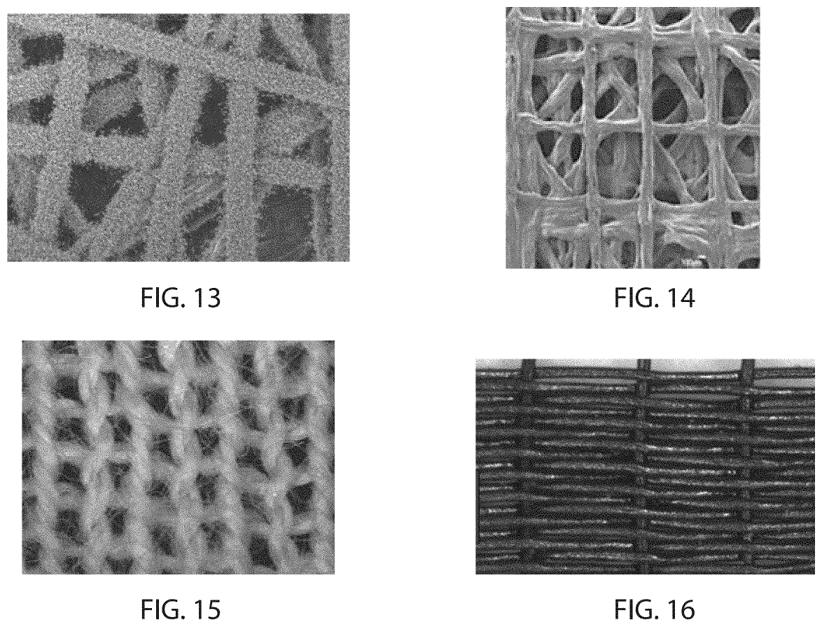

FIG. 13 illustrates a carbon nanotube textile formed by a plurality of carbon nanotube fibers and/or strands (e.g., those exemplified in FIGS. 9-12). The textile may be in the form of a random mesh arrangement, wherein fibers and/or strands of carbon nanotubes are configured randomly throughout the textile. FIG. 14 illustrates a carbon nanotube textile formed by a plurality of carbon nanotube fibers and/or strands in the form of a grid arrangement. For example, the textile of FIG. 14 may incorporate carbon nanotube fibers and/or strands running in approximately parallel paths, perpendicular paths, and/or at predefined angles. In another example, the textile of FIG. 14 may incorporate carbon nanotube fibers and/or strands arranged in a plurality of sheets, with the plurality of sheets stacked together to form the textile. A person of ordinary skill in the art will appreciate that various other textile weaves and arrangements may be possible to achieve desired characteristics.

FIG. 15 illustrates a textile formed of a fibrous material (e.g., cotton). For example, the textile may have a particular weave (e.g., jersey knit) to allow for stretchability, flexibility, and/or other desired properties. In another example, carbon nanotubes may be incorporated into, around, or otherwise with the fibrous material (e.g., forming a composite strand of carbon nanotube and fibrous material). FIG. 16 illustrates a textile formed of a fibrous material (e.g., polyester). A person of ordinary skill in the art will appreciate that additional weaves and configurations of composite fibers and/or strands may be utilized to achieve desired characteristics in accordance with the present invention.

Other aspects and embodiments of the present invention will be apparent to those skilled in the art from consideration of the specification and practice of the invention disclosed herein. It is intended, therefore, that the specification and illustrated embodiments be considered as examples only, with a true scope and spirit of the invention being indicated by the following claims.

What is claimed is:

1. A sleep system comprising:
a first textile including carbon nanotube material, wherein the textile is configured to exchange free-moving electrons with an organic body.

2. The sleep system of claim 1, wherein the first textile further includes fibrous material.

3. The sleep system of claim 2, wherein the first textile is comprised of about 30% carbon nanotube material and about 70% fibrous material.

4. The sleep system of claim 2, wherein the first textile is comprised of about 70% carbon nanotube material and about 30% fibrous material.

5. The sleep system of claim 1, wherein the sleep system further includes a second textile including carbon nanotube material.

6. The sleep system of claim 5, wherein the second textile further includes fibrous material.

7. The sleep system of claim 5, wherein the first and second textiles substantially enclose the body.

8. The sleep system of claim 1, wherein the first textile is coupled to a relatively greater negative electrical potential.

9. The sleep system of claim 1, wherein the first textile is coupled to a medical device capable of detecting the exchange of free-moving electrons.

10. The sleep system of claim 1, wherein the first textile is coupled to a local device, the local device coupled to a remote electronic device, and the remote electronic device capable of detecting the exchange of free-moving electrons.

11. A method comprising:
placing an organic body on a first textile, the first textile including carbon nanotube material; and
exchanging free-moving electrons between the organic body and the first textile.

12. The method of claim 11, further comprising:
exchanging free-moving electrons between the first textile and a relatively greater negative electrical potential.

13. A method comprising:
placing an organic body on a first textile, the first textile including carbon nanotube material; and
detecting the exchange of free-moving electrons between the organic body and the first textile.

14. The method of claim 13, further comprising:
viewing and/or interacting with the exchange of free-moving electrons via a user interface.

15. The method of claim 13, further comprising:
storing data corresponding to the exchange of free-moving electrons as detected.

16. The method of claim 15, further comprising:
viewing and/or interacting with the stored data via a user interface.

17. The method of claim 13, further comprising:
using the detected exchange of free-moving electrons to determine at least one biological process of the organic body; and
storing data corresponding to the at least one biological process.

18. The method of claim 15, further comprising:
viewing and/or interacting with the stored data via a user interface.

19. The method of claim 13 further comprising:
transmitting data corresponding to the exchange of free-moving electrons to a remote electronic device.

20. The method of claim 19, further comprising:
evaluating a health condition of the organic body based on the transmitted data.
